(19) United States
(12) Patent Application Publication
(10) Pub. No.: US 2023/0191083 A1
(43) Pub. Date: Jun. 22, 2023

(54) METHOD FOR CALCULATING THE PROXIMAL AND DISTAL ENDS OF AN INTERLACED DEVICE BEFORE BEING POSITIONED IN A VASCULAR STRUCTURE AND COMPUTER PROGRAMS THEREOF

(71) Applicants: Galgo Medical, SL, Barcelona (ES); Universidad Nacional Del Centro De La Provincia De Buenos Aires, Buenos Aires (AR)

(72) Inventors: Romina Luciana Munoz, Buenos Aires (AR); Ignacio Larrabide, Buenos Aires (AR); Hector Fernandez Martinez, Barcelona (ES)

(73) Assignees: Galgo Medical, SL, Barcelona (ES); Universidad Nacional Del Centro De La Provincia De Buenos Aires, Buenos Aires (AR)

(21) Appl. No.: 17/905,070

(22) PCT Filed: Oct. 6, 2020
(86) PCT No.: PCT/EP2020/077935

§ 371 Date: Aug. 26, 2022

(30) Foreign Application Priority Data
Feb. 28, 2020 (EP) 20382143.4

(51) Int. Cl. A61M 25/01

(52) U.S. Cl. CPC A61M 25/01; A61M 2025/09175

(57) ABSTRACT

A method and computer program for calculating proximal and distal ends of an interlaced device before being positioned in a vascular structure are proposed. The method comprises receiving a three-dimensional image of a vascular structure in which an interlaced device with a singularity at a proximal and/or distal end will be positioned. A central line of said structure which defines a direction in which the interlaced device is to be deployed is traced. A point P_{d} on the traced central line and the local morphology of the vessel are defined, wherein point P_{d} indicates the point where the distal end of the interlaced device will start to be deployed. A proximal point Pₚ is calculated using the distal point P_{d} and the local morphology of the vessel, both having been defined. The proximal and distal ends are calculated depending on if the singularity is at the proximal and/or distal ends.

### TECHNICAL FIELD

The present invention relates to a computational method and computer products for calculating the proximal and distal ends of a device of interlaced threads (or interlaced device) with one or more sections with a singularity, before being positioned in a vascular structure. The invention allows knowing the configuration the interlaced device will adopt before it is implanted. The invention also allows calculating the local porosity of the device after it is implanted/positioned.

By singularity it should be understood a point, or section, along the longitudinal direction of the interlaced device, where multiple (at least three) of the interlaced filaments/threads coincide. That is, because of the singularity/singularities the interlaced device is “closed” at some point where (all or at least most of) the threads converge. The device can be closed at one of its ends, at both ends, or in more sites.

### BACKGROUND OF THE INVENTION

Examples of interlaced devices are those used in the treatment of vascular pathologies when deployed inside a vessel. More particularly, examples of devices of this type are the intrasaccular devices described in patent application US 20120283768-A1 and in U.S. Pat. No. 10,136,896-B2.

Likewise, a tool which allows a user to manually position a device with singularities inside vascular models, but without simulating the deployment method is disclosed in papers [1] and [2]. This tool is based on deformation by means of a spring model.

Furthermore, U.S. Pat. No. 10,176,566-B2 proposes a method for determining the final length of a stent before the positioning thereof in a vascular structure. The present invention extends and adapts what is described in this US patent.

Likewise, the US 2019038358-A1 provides a method of estimating the length of a stent. Unlike present invention, the stent of this US patent application does not have any singularity in the end sections thereof.

Therefore, new methods which allow calculating the final positions of interlaced devices with one or more singularities are required.

### REFERENCES

[1] J R Cebral, et al. Analysis of flow dynamics and outcomes of cerebral aneurysms treated with intrasaccular flow-diverting devices. American Journal of Neuroradiology, 2019.
[2] Fernando Mut, et al. Image-based modeling of blood flow in cerebral aneurysms treated with intrasaccular flow diverting devices. International journal for numerical methods in biomedical engineering, page e3202, 2019.

### DESCRIPTION OF THE INVENTION

To that end, embodiments of the present invention provide according to a first aspect a method for calculating the proximal and distal ends (i.e. the final positions) of an interlaced device before being positioned in a vascular structure. The method comprises performing the following steps by a computer including one or more processors and at least one memory:
receiving a three-dimensional image of a vascular structure in which the device formed by interlaced threads (hereinafter interlaced device) will be positioned, and tracing a central line of said vascular structure in the three-dimensional image which defines a direction in which the interlaced device is to be deployed, wherein the interlaced device includes at a proximal end or at a distal end thereof a section with a singularity (i.e. a zone where a plurality of the interlaced threads coincide);
defining, based on an input provided by a user, a distal point P_{d} on the traced central line and a local morphology of the vessel (i.e. the shape of the vessel in the vicinity of a given point), wherein the distal point P_{d} indicates the point where the distal end of the interlaced device will start to be deployed; and
calculating a proximal point Pₚ using the distal point P_{d} and the local morphology of the vessel, both having been defined, wherein the proximal point Pₚ indicates the point that limits a portion of the central line over the traced central line that will be needed for deploying the section of the interlaced device including the singularity.

If the singularity of the interlaced device is at the distal end, the method further comprises extracting a morphological descriptor m_{d} of the vascular structure at the distal point P_{d} and comparing said morphological descriptor m_{d} with a nominal morphological descriptor Mₙ of a distal section of the interlaced device including the singularity. Then, a point Pₐ is defined as Pₐ=P_{d}.

In the event that the morphological descriptor m_{d} is smaller than the nominal morphological descriptor Mₙ, the method further comprises:
i. making the point Pₐ equal to a point next to Pₐ in a proximal direction along the traced central line,
ii. calculating a local morphological descriptor mₐ of a cross-section of the vascular structure at said point Pₐ,
iii. calculating a distance hₐ as hₐ=Mₙ·dumping (mₐ), where dumping(m) is a mathematical function in the interval [0,1] which considers the variation of hₐ according to the expansion of said distal section of the interlaced device including the singularity to an expansion diameter corresponding to the morphological descriptor mₐ,
iv. identifying a point Pₐₘ that is located an interval (or length) hₐ from the distal point P_{d} and on a plane perpendicularly intersecting the traced central line at point Pₐ,
v. calculating d_{d} as the distance between the point Pₐ and the point Pₐₘ, and
vi. comparing the calculated distance d_{d} with the local morphological descriptor mₐ, wherein:
if the distance d_{d} is smaller than the local morphological descriptor mₐ, steps i. to v. are repeated, and if the distance d_{d} is greater than or equal to the local morphological descriptor mₐ, Pₚ=Pₐ is defined as the proximal point that limits a portion of the central line over the traced central line that will be needed for deploying the distal section of the interlaced device including the singularity.

In the event that the morphological descriptor m_{d} is greater than or equal to the nominal morphological descriptor Mₙ, the method comprises selecting the proximal point Pₚ as the point that is located a distance dₘᵢₙ from the distal point P_{d} in the proximal direction, where dₘᵢₙ is the minimum height, over the traced central line, achieved by said distal section, corresponding to the height of the distal section being in the configuration corresponding to the nominal morphological descriptor Mₙ (i.e. the nominal configuration, where the interlaced device is open to its maximum diameter, without constraints. Indeed, dₘᵢₙ is a constant determined by the design of the device).

In contrast, if the singularity is at the proximal end, the method further comprises:
extracting a morphological descriptor m_{d} of the vascular structure at the distal point P_{d};
calculating a distance h_{d} as h_{d}=Mₙ dumping (m_{d}), where dumping(m) is a mathematical function in the interval [0,1] which considers the variation of h_{d} according to the expansion of a proximal section of the interlaced device including the singularity to an expansion diameter corresponding to the morphological descriptor m_{d}; and
comparing said morphological descriptor m_{d} with a nominal morphological descriptor Mₙ of the proximal section of the interlaced device including the singularity.

Then, a point Pₐ is defined as Pₐ=P_{d}, and a point P_{dm} that is located an interval m_{d} from the distal point P_{d} and on a plane perpendicularly intersecting the traced central line at the distal point P_{d} is identified.

In the event that the morphological descriptor m_{d} is smaller than the nominal morphological descriptor Mₙ, the method further comprises:
vii. making the point Pₐ equal to a point next to Pₐ in a proximal direction along the traced central line,
viii. calculating dₐ as the distance between the point Pₐ and the point P_{dm}, and
ix. comparing the calculated distance dₐ with the distance h_{d}, wherein:
if the distance dₐ is smaller than the h_{d}, steps vii. and viii. are repeated, and if the distance dₐ is greater than or equal to the distance h_{d}, Pₚ=Pₐ is defined as the proximal point that limits a portion of the central line over the traced central line that will be needed for deploying the proximal section of the interlaced device including the singularity.

In the event that the morphological descriptor m_{d} is greater than or equal to the nominal morphological descriptor Mₙ, the method comprises selecting the proximal point Pₚ as the point that is located a distance dₘᵢₙ from the distal point P_{d} in the proximal direction, where dₘᵢₙ is the minimum height, over the traced central line, achieved by said proximal section, corresponding to the height of said proximal section being in the configuration corresponding to the morphological descriptor m_{d}.

In one embodiment, the interlaced device has a singularity in the proximal section and a singularity in the distal section, therefore the calculation of the proximal and distal ends of the interlaced device is performed for both proximal and distal sections, according to the steps described above. In this case, however, the distal point P_{d} of the proximal section is defined as the proximal point Pₚ of the distal section.

In one embodiment, the interlaced device further has a (tubular-shaped) central section. The traced central line of the vascular structure is also divided into different segments. The method in this case further comprises:
x. selecting a point P_{c} of the central line at which the deployment of the device of the distal section ended, wherein the point P_{c} is the proximal point Pₚ of the distal section;
xi. extracting from the central line at least one morphological descriptor m_{c} of the segment corresponding to point P_{c};
xii. calculating a height of the interlaced device for a first segment using a ratio indicating a change in height of the interlaced device according to the local morphology of the vascular structure;
xiii. subtracting said calculated height from a nominal height of the interlaced device, obtaining a new nominal height,

wherein if said new nominal height is greater than 0, steps xi) to xiii) are repeated for the segment contiguous to the preceding segment, moving forward in the proximal direction, and if the new nominal height is approximately 0, all the lengths of each segment are added together, this sum being the final height of the interlaced device after its positioning.

In one embodiment, the interlaced device is attached to a second interlaced device (of the same diameter) at either the proximal end or distal end thereof. The second interlaced device can include a singularity in its proximal section, in its distal section, or in both. In this case, the proposed method comprises performing the calculation of the proximal and distal ends of the second interlaced device according to the steps described above, depending on where the singularity/singularities is/are located.

According to the proposed method, the morphological descriptors m_{d} and mₐ can include: the minimum radius of the cross-section of the vasculature perpendicular to the central line, or the maximum radius, or the radius of the equivalent circumference with the perimeter equal to the cross-section perpendicular to the central line, or the radius of the equivalent circumference with an area equal to the cross-section perpendicular to the central line, among others.

In one embodiment, the method further comprises calculating the porosity on the entire surface of the interlaced device. To that end, the method comprises:
dividing the surface of the singularity into a specific number of portions, wherein said number of portions have a common center at the distal point P_{d}, cover the entirety of said surface, and depend on the number of interlaced threads constituting the interlaced device;
dividing the surface of the singularity into concentric circumferences, wherein said circumferences have a common center at the distal point P_{d};
dividing the surface into a plurality of cells, wherein each cell is obtained considering the section of one of said portions contained between two consecutive concentric circumferences;
calculating for each of the plurality of cells: the total area, the area of the thread going through the cell and the uncovered area; and
calculating the porosity of each cell as the ratio between the uncovered area divided by the total area of the cell or the ratio between the covered area divided by the total area of the cell.

In the previous embodiment, all the portions can have the same or a different size. In particular, the number of portions is equal to the number of threads.

Other embodiments of the invention disclosed herein also include computer program products for performing the steps and operations of the proposed method in the first aspect of the invention. More particularly, a computer program product is an embodiment having a computer-readable medium including computer program instructions encoded therein which, when executed in at least one processor of a computer system, cause the processor to perform the operations indicated herein as embodiments of the invention.

The present invention thus provides a fast method to simulate intra-sacular devices inside the patient anatomy that allows knowing, in a quick and precise way, how a specific device size adapts to the aneurysm morphology. That is, the present invention allows knowing, before being implanted, the final arrangement of a device with singularities when it is positioned inside an aneurysm. This involves, in addition to the possibility of evaluating the apposition with respect to the walls of the aneurysm achieved by the device, the location of the proximal end thereof inside the aneurysm. This location is important because it helps the neurointerventionalist to make decisions about the device that is to be used. The device is ideally expected to completely and safely cover the neck of the aneurysm to interrupt the blood flow inside the aneurysm.

### BRIEF DESCRIPTION OF THE DRAWINGS

The foregoing and other features and advantages will be better understood from the following merely illustrative and non-limiting detailed description of several embodiments in reference to the attached drawings, in which:

FIG. 1 schematically illustrates an interlaced device divided into three sections, according to an embodiment of the present invention.

FIG. 2 is a flowchart illustrating a method for calculating the proximal and distal ends of an interlaced device before being positioned in a vascular structure, according to an embodiment of the present invention.

FIG. 3 is a flowchart illustrating a method for calculating the proximal and distal ends of an interlaced device before being positioned in a vascular structure when the interlaced device includes a singularity at its distal end, according to an embodiment of the present invention.

FIG. 4 is a flowchart illustrating a method for calculating the proximal and distal ends of an interlaced device before being positioned in a vascular structure when the interlaced device includes a singularity at its proximal end, according to an embodiment of the present invention.

FIG. 5 is a flowchart illustrating the calculation for the central section of the interlaced device, according to an embodiment of the present invention.

FIG. 6 illustrates the step-by-step virtual deployment of the interlaced device inside a vascular structure. FIG. 6A shows the extraction of the central line; FIG. 6B shows the deployment of the distal section with a singularity; FIG. 6C shows the deployment of the central section next to the previously deployed section; FIG. 6D shows the deployment of the proximal section with a singularity next to the previously deployed sections, imparting the final configuration of the device.

FIG. 7 is a flowchart illustrating a method for calculating the porosity of an interlaced device with one or more singularities, according to an embodiment of the present invention.

FIGS. 8A-8C graphically show some of the steps implemented by the proposed method for calculating the porosity of an interlaced device with one or more singularities, according to an embodiment of the present invention.

FIGS. 9A-9C illustrate an example of how the calculation of the total area is performed, the area of the thread going through a cell, and the uncovered area for each of the cells.

### DETAILED DESCRIPTION OF PREFERRED EMBODIMENTS

The present invention provides a method for calculating the final position of interlaced devices with one or more singularities. The selection of an appropriate endosaccular device size is crucial for a successful treatment and strongly depends of the final configuration that the device adopts when it adapts to the aneurysm sac morphology. This is frequently a problem during the intervention, leading to replacement of the device, reopening of the aneurysm or a need for re-treatment. A technique that allows predicting the released device configuration before intervention provides a powerful computational tool to aid the interventionist during device selection.

The method is based on the analysis of the local morphology of the region to be treated, more specifically on the analysis of how the interlaced device is deformed as it adapts to the local morphology of the vessel, for example an aneurysm. The morphological description of the aneurysm and the descriptive specifications of the interlaced device design (height, diameter, amount of threads, etc.) are also preferably taken into account by the proposed method.

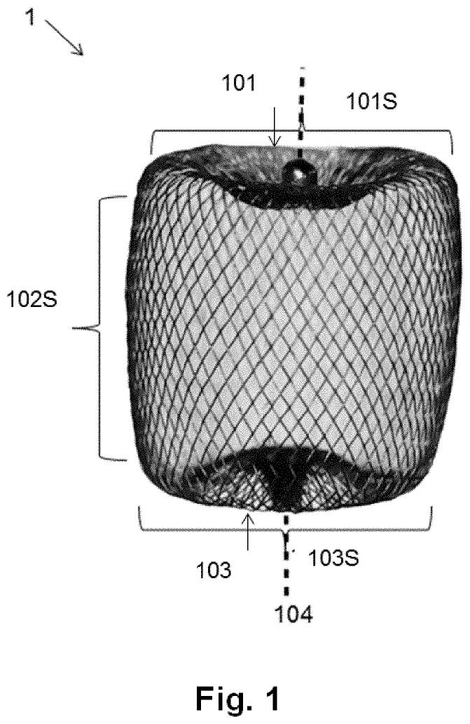

In reference to FIG. 1, said figure shows an example of an interlaced device 1 with three different sections, a proximal section 103S with a singularity (even though the threads are not observed), a central 102S tubular-shaped and a distal section 101S with a singularity (even though the threads are not observed).

The interlaced device 1 of FIG. 1 is similar to a WEB (Woven Endo Bridge) type device such as the one described in documents US 20120283768-A1 and U.S. Pat. No. 10,136,896-B2. In this particular case, the interlaced device 1 is closed at both ends and the interlaced device is considered to be characterized by said three sections 101S, 102S, 103S.

It should be noted that the proposed method is generic and can be simplified to as many sections as desired; in fact, it is only necessary for one of the ends 101, 103 of the interlaced device 1 to include a singularity. The interlaced device 1 can also be attached to a second interlaced device through the proximal end 103 or distal end 101.

In this case, it is only necessary for contiguous sections to coincide with one another in type (singularity or open) and in size (the same diameters). The interlacing configuration and design that the threads of the interlaced device 1 have in each of its sections, particularly in the proximal and distal sections 103S, 101S, can thereby especially be considered.

In the interlaced device 1, the threads in the sections 101S, 103S with singularities extend radially, all of said threads being attached to a point referred to as the hub, located on axis 104 of the interlaced device 1. The threads in these sections 101S, 103S are constituted by adopting a sinusoidal shape in the radial direction when the interlaced device is deployed outside the catheter. The position of the threads in the radial direction can be exemplified using a sinusoidal function for the interlaced device 1. Nevertheless, other functions such as a sinusoidal function with an exponential decay, a logarithmic function, a catenary function, an exponential function, etc., could also be used.

Local morphology of the vessel can be quantified using morphological descriptors that are computed semi-automatically using state-of-the-art software.

In this description, the term “nominal morphological descriptor Mₙ” is used to refer to the magnitude achieved by a descriptor of the morphology of the interlaced device 1, such as radius or height, when the device is released outside of a vascular structure or of the positioning device (catheter). This configuration of the interlaced device 1 is referred to as the nominal (free) configuration. Therefore, for example, if the radius is considered as a morphological descriptor of the interlaced device 1, the nominal radius is the radius it will adopt when it is completely free, coinciding with the maximum radius it may reach.

On the other hand, the morphological descriptors m_{d} and mₐ (and also m_{c} if this descriptor is calculated) can include any of: the minimum radius of the cross-section of the vasculature perpendicular to the central line, the maximum radius, the radius of the equivalent circumference with the perimeter equal to the cross-section perpendicular to the central line, the radius of the equivalent circumference with an area equal to the cross-section perpendicular to the central line, etc., or combinations of the foregoing.

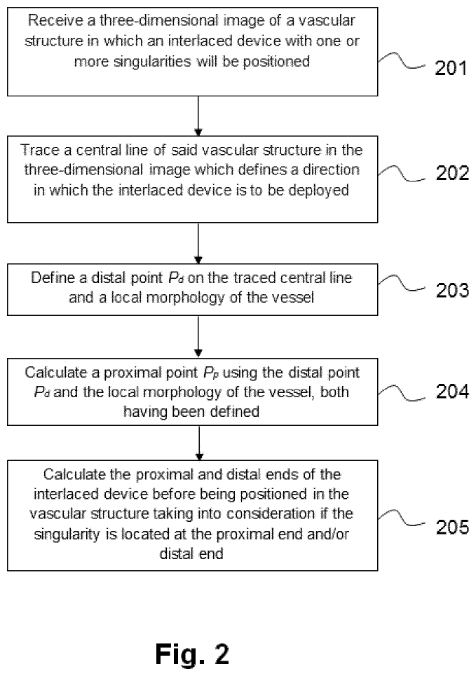

FIG. 2 illustrates an embodiment of a method for calculating the proximal and distal end of an interlaced device before being positioned in a vascular structure. The method in step 201 comprises receiving a three-dimensional image of a vascular structure in which a device formed by interlaced threads, or interlaced device, with one or more singularities (for example the interlaced device 1 of FIG. 1), will be positioned. In step 202, the method comprises tracing a central line of said vascular structure in the three-dimensional image which defines a direction in which the interlaced device 1 is to be deployed. This direction may or may not be the main direction of the vessel (or aneurysm), according to the configuration thereof and the manner of accessing same with the catheter. Then, in step 203, the method comprises defining a distal point P_{d} on the traced central line and a local morphology of the vessel. The distal point P_{d} indicates the point where the distal end 101 of the interlaced device 1 will start to be deployed. In step 204, a proximal point Pₚ is calculated using the distal point P_{d} and the local morphology of the vessel, both having been defined. The proximal point Pₚ indicates the point that limits a portion of the traced central line over the traced central line that will be needed for deploying the mentioned section of the interlaced device including the singularity. Finally, in step 205, the proximal and distal ends of the interlaced device 1 are calculated taking into consideration whether the singularity is located at the proximal end 103 and/or distal end 101.

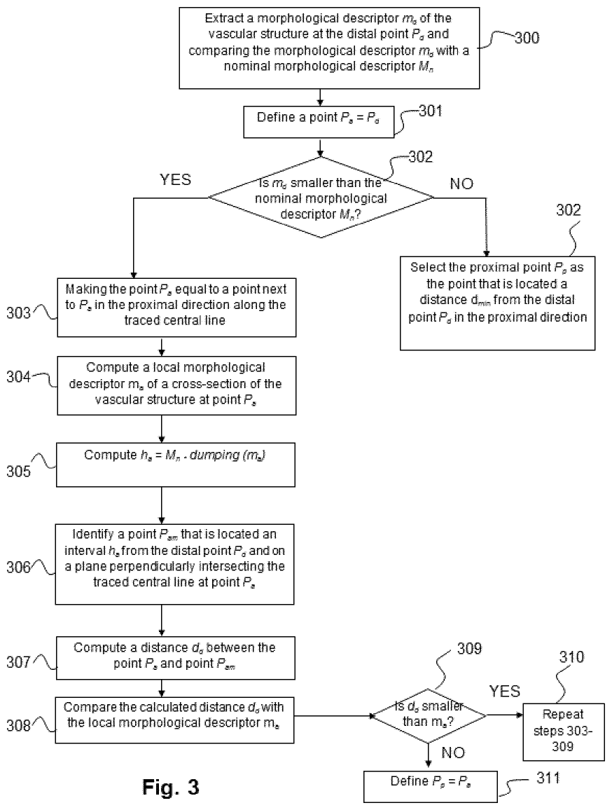

FIG. 3 illustrates an embodiment of the calculation of the singularity if the latter is located in the distal section of an interlaced device. The method comprises extracting (step 300) a morphological descriptor m_{d} of the vascular structure at the distal point P_{d} and comparing the morphological descriptor m_{d} with a nominal morphological descriptor Mₙ of the distal section 101S of the interlaced device 1. At step 301, a point Pₐ is defined as Pₐ=P_{d}.

If the morphological descriptor m_{d} is smaller than the nominal morphological descriptor Mₙ, the method further comprises (step 303) making the point Pₐ equal to a point next to Pₐ in the proximal direction along the traced central line, calculating (step 304) a local morphological descriptor mₐ of a cross-section of the vascular structure at said point Pₐ, and calculating (step 305) hₐ=Mₙ·dumping (mₐ), where hₐ refers to a distance, and dumping(m) is a mathematical function in the interval [0,1] that accounts for the variation of hₐ when the interlaced device 1 has different expansion diameters (or simply expansions).

The relation of the device expansion and the height of the distal section 101S having the singularity is nonlinear. Thus, the damping function accounts for this nonlinearity by “damping” (i.e. multiplying by a number between 0 and 1) the nominal morphological descriptor Mₙ to obtain the height hₐ. In the case of a linear relation between the device expansion and its height, the damping will be constant for different values of mₐ. It might also be equal to 1, depending on the design and behavior of the interlaced device 1. Consequently, the expansion diameter (or simply expansion) is the diameter achieved by the interlaced device 1 and can go from 0 (the interlaced device 1 is fully closed, e.g. inside the catheter) to the nominal diameter (i.e. the diameter of the interlaced device 1 when is fully opened, with the maximum diameter). In this particular embodiment, the expansion diameter is governed by mₐ. The larger the vessel is the larger mₐ and the larger expansion will be, but not being larger than the nominal diameter.

In step 306, the method comprises identifying a point Pₐₘ that is located an interval hₐ from the distal point P_{d} and on a plane perpendicularly intersecting the traced central line at point Pₐ.

In step 307, a distance d_{d} between the point Pₐ and point Pₐₘ is calculated, and the calculated distance d_{d} is compared (step 308) with the local morphological descriptor mₐ. If d_{d} is smaller (step 310) than the local morphological descriptor mₐ, steps 303-309 are repeated. Otherwise, (step 311) Pₚ=Pₐ is defined as the proximal point that limits a portion of the central line over the traced central line that will be needed for deploying the distal section 101S of the interlaced device 1.

It should be noted that the point Pₐ refers to the current point being studied at a given iteration of the algorithm, as a candidate to release the distal section 101S. The point Pₐ is iteratively searched, along the traced central line, advancing one small step at a time in the proximal direction. Because Pₐ candidates are predefined in some embodiments it can happen that a point Pₐ satisfying d_{d}=mₐ might not exist. So, associated Pₐₘ to Pₐ should be such that the d_{d}=>hₐ in the iterative search.

If the morphological descriptor m_{d} is greater than or equal to the nominal morphological descriptor Mₙ, the method comprises (step 302) selecting the proximal point Pₚ as the point that is located a distance dₘᵢₙ from the distal point P_{d} in the proximal direction, where dₘᵢₙ is the minimum height, in the direction of the traced central line, achieved by said distal section 101S of the interlaced device 1, corresponding to the height of the section 101S being in the configuration corresponding to the nominal morphological descriptor Mₙ.

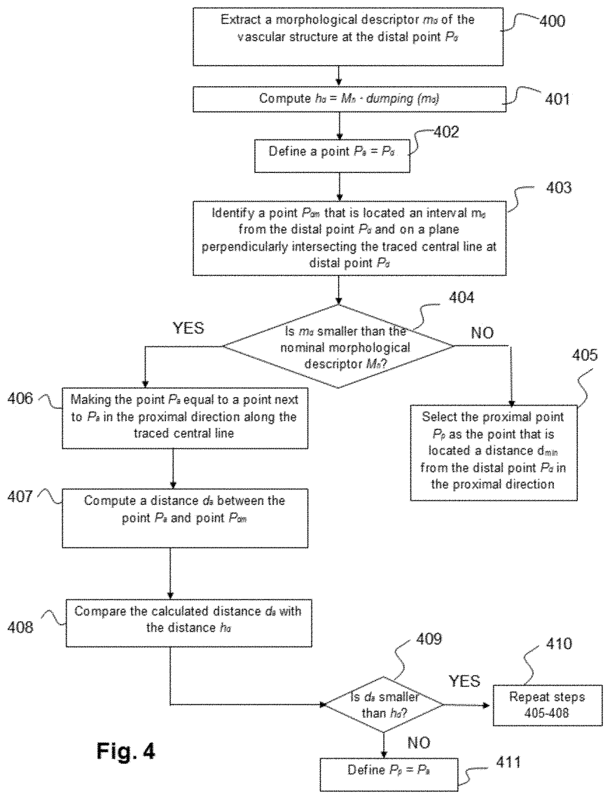

FIG. 4 illustrates an embodiment of the calculation of the singularity if the latter is in the proximal section of an interlaced device. In step 400, the method comprises extracting a morphological descriptor m_{d} of the vascular structure at the distal point P_{d}. Then, in step 401, h_{d}=Mₙ·dumping (m_{d}) is calculated, where h_{d} is a distance, and dumping(m) is a mathematical function in the interval [0,1] which in this case considers the variation of h_{d} according to the expansion of the proximal section 103S of the interlaced device 1 (i.e. the section having the singularity in this case), to an expansion diameter corresponding to the morphological descriptor m_{d}. Different to the embodiment of FIG. 3, in this case, the expansion diameter is governed by m_{d}.

In step 402, a point Pₐ is defined as Pₐ=P_{d}. Then, in step 403, the method comprises identifying a point P_{dm} that is located an interval m_{d} from the distal point P_{d} and on a plane perpendicularly intersecting the traced central line at the distal point P_{d}.

In step 404, the method comprises comparing the morphological descriptor m_{d} with a nominal morphological descriptor Mₙ.

If the morphological descriptor m_{d} is smaller than the nominal morphological descriptor Mₙ, the method comprises making (step 406) the point Pₐ equal to a point next to Pₐ in the proximal direction along the traced central line, calculating (step 407) a distance dₐ between point Pₐ and point P_{dm}, and comparing (step 408) the calculated distance dₐ with the distance h_{d}.

If the distance dₐ is smaller than the distance h_{d} (step 409), the preceding steps 406-408 are repeated (step 410). Otherwise, in step 411, Pₚ=Pₐ is defined as the proximal point that limits a portion of the central line over the traced central line that will be needed for deploying the proximal section 103S of the interlaced device 1.

As outlined for the section with a singularity at the distal end 101, it should be noted that the point Pₐ refers to the current point being studied at a given iteration of the algorithm, as a candidate to release the distal section 101S. The point Pₐ is iteratively searched, along the traced central line, advancing one small step at a time in the proximal direction. Because Pₐ candidates are predefined in some embodiments it can happen that the point Pₐ satisfying that dₐ=h_{d} might not exist. So, associated P_{dm} to Pₐ should be such that dₐ=>h_{d} in the iterative search.

If the morphological descriptor m_{d} is greater than or equal to the nominal morphological descriptor Mₙ (step 404), the method comprises selecting the proximal point Pₚ as the point that is located a distance dₘᵢₙ from the distal point P_{d} in the proximal direction, where dₘᵢₙ is the minimum height, over the traced central line, achieved by the proximal section 103S, corresponding to the height of said section 103S being in the configuration corresponding to the morphological descriptor m_{d}.

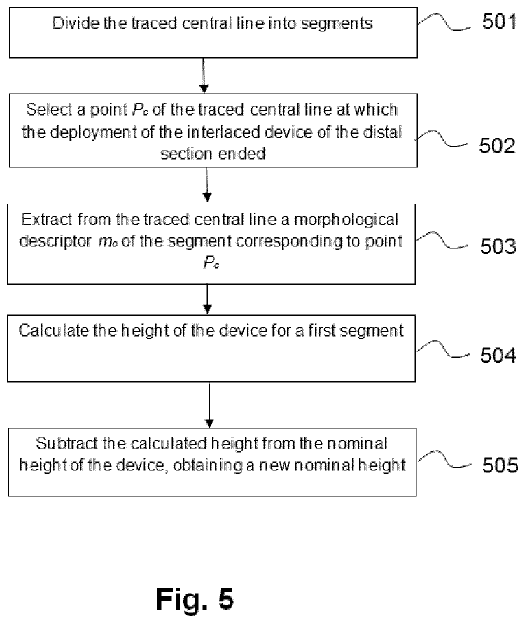

In the event that the interlaced device is identical to the device of FIG. 1 and includes a tubular-shaped central section, the methodology to be implemented according to one embodiment would be the one described in FIG. 5. First the traced central line of the vascular structure is divided into different segments (step 501). Then, in step 502, a point P_{c} of the traced central line at which deployment of the interlaced device 1 of the distal section 101S ended is taken, and one or more morphological descriptors m_{c} of the segment corresponding to point P_{c} is/are extracted (step 503) from the traced central line. Next, in step 504, the height of the interlaced device 1 for a first segment is calculated, particularly using an indicator ratio. The indicator function determines a change in height of the interlaced device 1 according to the local morphology of the vascular structure. This morphological information is obtained from the description of the interlaced device design, for example, amount of threads, interlacing angle, length of the threads, height and diameter of the interlaced device deployed outside of the vessel, etc.). In step 505, the calculated height is subtracted from the nominal height of the interlaced device 1, obtaining a new nominal height that will be used in the following iteration of the method.

If the new nominal height is greater than 0, steps 503 to 505 are repeated for the segment contiguous to the preceding segment, moving forward in the proximal direction. If the new nominal height is approximately 0 (smaller than the separation between points of the traced central line), the lengths of all the segments in respect of which it moved forward are added together, this sum being the final height of the interlaced device 1 after its positioning.

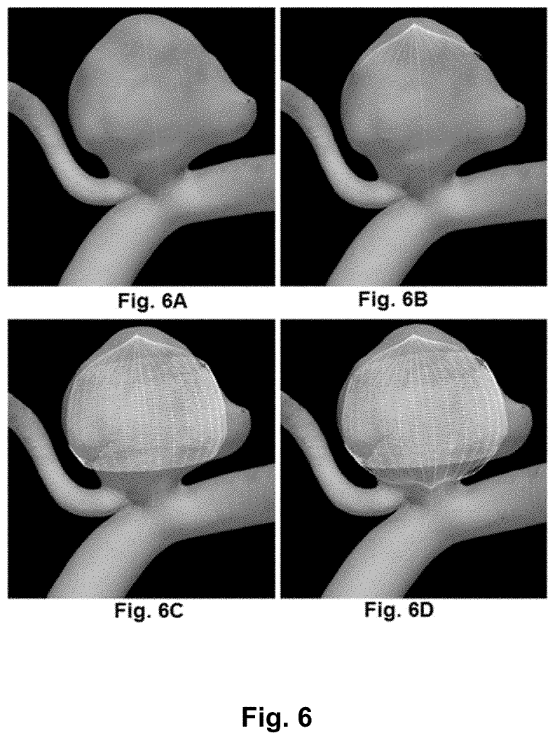

FIG. 6 illustrates the result obtained upon application of the proposed method. In FIG. 6D, the final arrangement of the interlaced device 1 implanted in the vascular structure can be observed.

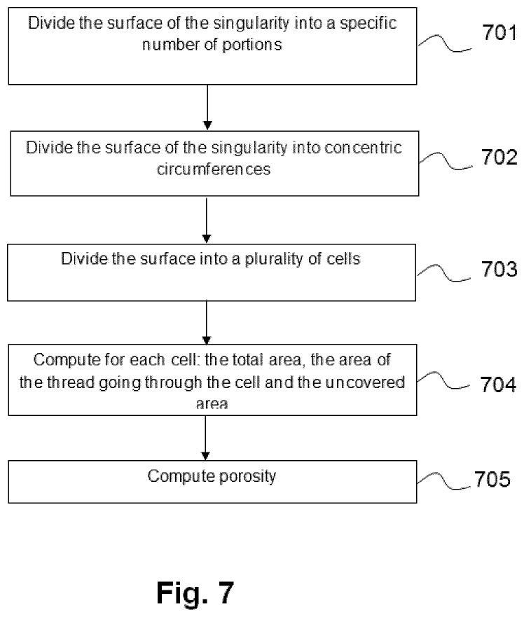

FIG. 7 shows an embodiment of the calculation of the porosity of the interlaced device 1, with one or more of its ends closed, not only on the sides thereof but also at the sections (singularities). In step 701, the method comprises dividing the surface of the singularity into a specific number of portions, or unitary portions 81. The number of portions 81 has a common center 80 at the distal point P_{d}, cover the entire mentioned surface, and depend on the number of interlaced threads constituting the interlaced device 1. In one embodiment, the surface of the singularity is Nₕ, where Nₕ is the total number of threads constituting the device, obtaining the surface of the singularity partitioned into straight lines converging at the hub, equidistant at an angle

$$Δ θ = \frac{2 π}{{N}_{h}} .$$

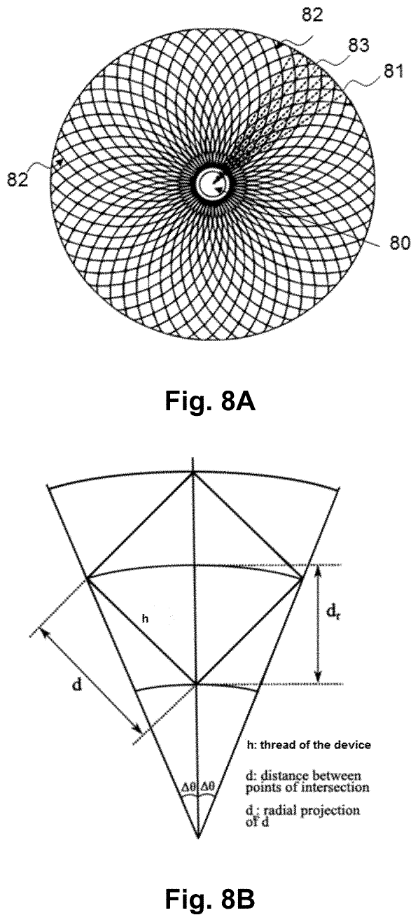

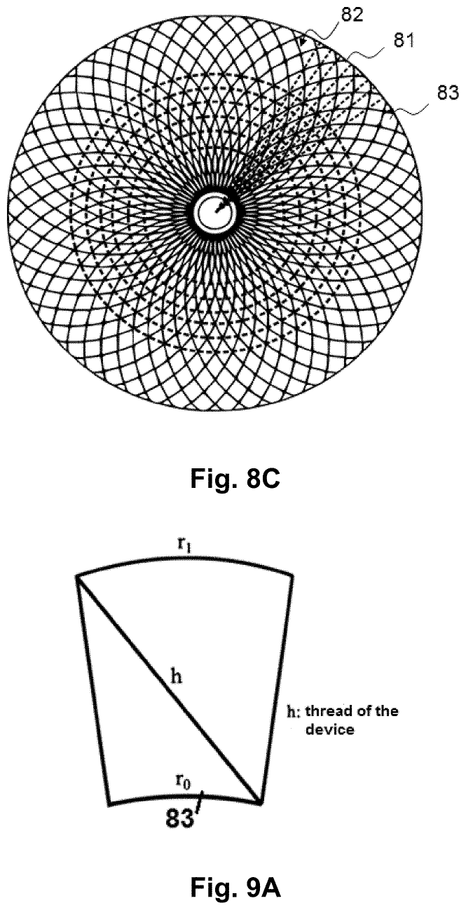

In step 702, the method comprises dividing the surface of the singularity into concentric circumferences 82, wherein the circumferences have a common center at the distal point P_{d}, and subsequently dividing (step 703) the surface into a plurality of cells 83. Each cell is obtained particularly considering the section of one of said portions 81 contained between two consecutive concentric circumferences 82. FIG. 8A graphically shows the preceding steps 701-703.

FIG. 8B illustrates the ratio of the radii of the circumference of FIG. 8A, the radii being equal to the radial projection dᵣ of the distance d between points of intersection. Concentric rings partitioned by the straight lines converging at the hub considered in step 701 are thereby obtained (see FIG. 8C). Each of the partitions represents a cell 83. In one embodiment, the polar coordinate system is used with the pole being the hub of the interlaced device 1 and the polar axis being one of the converging straight lines.

Continuing with the methodology of FIG. 7, in step 704, the method comprises calculating for each cell 83: the total area, the area of the thread going through the cell 83, and the uncovered area for each of the cells 83.

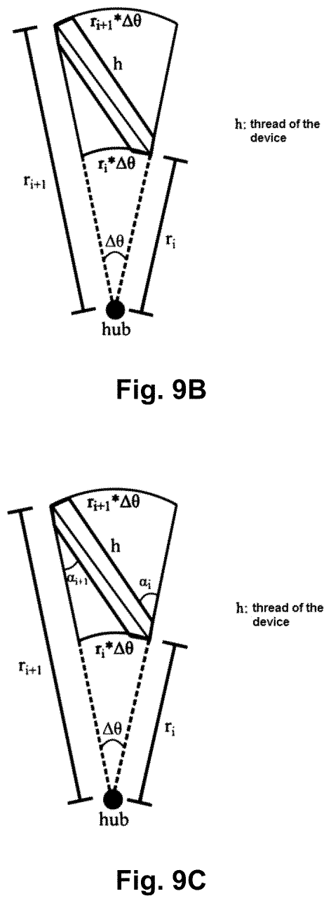

FIGS. 9A-9C illustrate an example of performing the calculation of the total area, the area of the thread going through the cell 83, and the uncovered area for each of the cells 83. To that end, as shown in FIG. 9A, the circumference of radius r₁=dᵣ_{₁} is considered. In this initial step, r₀ is the circumference of the hub of the interlaced device 1. In polar coordinates, the area of the cell 83 is calculated (see FIG. 9B):

A_{c}=∫_{θ}_{₁}^{θ}^{₂}½(r₁²−r₀²)dθ

For the partition performed, the variation dθ of the angle is constant Δθ, then:

A_{c}=½(r₁²−r₀²)Δθ

In general for the cell i, i.e., for the circumference of radius rᵢ=dᵣ_{ᵢ}, the area is calculated:

A_{c}=½(rᵢ²−rᵢ₋₁²)Δθ

where rᵢ₋₁ is the radius of the circumference taken in step i−1 and constituting the lower section of the current cell. In one embodiment, in each concentric circumference 82 the total number of cells 83 is Nₕ and the area is the same in all of them. The area of the uncovered surface of the cell 83 depends on the angle α formed by the thread enclosed in cell 83 with the radial direction.

A_{uncovered}=A_{c}−Aₕ

where A_{c} is the total area of the cell 83 and Aₕ is the area of the thread inside the cell 83.

The area of the uncovered surface consists of two “triangles” the surfaces of which depend on angles αᵢ and αᵢ₋₁ (see FIG. 9C).

Finally, in step 705, the porosity of each cell 83 is calculated as the ratio between the uncovered area divided by the total area of the cell 83 or the ratio between the covered area divided by the total area of the cell 83.

The proposed invention can be implemented in hardware, software, firmware, or any combination thereof. If it is implemented in software, the functions can be stored in or encoded as one or more code instructions in a computer-readable medium.

The scope of the present invention is defined in the attached claims.

What is claimed is:

1. A computer-implemented method for calculating proximal and distal ends of an interlaced device before being positioned in a vascular structure, the computer-implemented method comprising:
using a computer to receive a three-dimensional image of a vascular structure in which a device formed by interlaced threads, also termed interlaced device, will be positioned, and tracing a central line of the vascular structure in the three-dimensional image defining a direction in which the interlaced device is to be deployed, the interlaced device comprising a proximal end disposed at a proximal section thereof, the proximal end comprising a singularity, the singularity comprising a coincidence of a plurality of interlaced threads, or the interlaced device comprising distal section disposed at a distal end thereof, the distal section comprising the singularity;
using the computer to define, based on an input provided by a user, a distal point P_{d} on the traced central line and a local morphology of a vessel, the distal point P_{d} being configured to indicate a point where the distal end will start to be deployed;
using the computer to calculate a proximal point Pₚ by using the defined distal point P_{d} and the defined local morphology of the vessel, the proximal point Pₚ being configured to indicate a point that limits a portion of the central line over the traced central line that will be needed for deploying the proximal section comprising the singularity, or that will be needed for deploying the distal section comprising the singularity:
if the distal section comprises the singularity, the method further comprises extracting a morphological descriptor m_{d} of the vascular structure at the distal point P_{d} and comparing the morphological descriptor m_{d} with a nominal morphological descriptor Mₙ of the distal section:
a point Pₐ being defined as Pₐ=P_{d};
if the morphological descriptor m_{d} is smaller than the nominal morphological descriptor Mₙ, the method further comprises:
i. making the point Pₐ equal to a point next to Pₐ in a proximal direction along the traced central line,
ii. calculating a local morphological descriptor mₐ of a cross-section of the vascular structure at the point Pₐ,
iii. calculating a distance hₐ as hₐ=Mₙ·dumping (mₐ), where dumping(m) is a mathematical function in an interval [0,1], which considers a variation of hₐ according to an expansion of the distal section to an expansion diameter corresponding to the local morphological descriptor mₐ,
iv. identifying a point Pₐₘ that is located an interval hₐ away from the distal point P_{d} and on a plane perpendicularly to and intersecting the traced central line at point Pₐ,
v. calculating d_{d} as the distance between the point Pₐ and the point Pₐₘ, and
vi. comparing the calculated distance d_{d} with the local morphological descriptor mₐ;
if the distance d_{d} is smaller than the local morphological descriptor mₐ, the method further comprises repeating steps i. to v.,
if the distance d_{d} is greater than or equal to the local morphological descriptor mₐ, Pₚ=Pₐ is defined as the proximal point that limits a portion of the central line over the traced central line that will be needed for deploying the distal section; or
if the morphological descriptor m_{d} is greater than or equal to the nominal morphological descriptor Mₙ, the method comprises selecting the proximal point Pₚ as the point that is located a distance dₘᵢₙ from the distal point P_{d} in the proximal direction, where dₘᵢₙ is a minimum height, over the traced central line, defined by the distal section, and corresponding to a height of the distal section being in a configuration corresponding to the nominal morphological descriptor Mₙ; or
if the singularity is at the proximal end, the method further comprises:
extracting a morphological descriptor m_{d} of the vascular structure at the distal point P_{d};
calculating a distance h_{d} as h_{d}=Mₙ·dumping (m_{d}),
where dumping(m) is a mathematical function in an interval [0,1] which considers a variation of h_{d} according to an expansion of the proximal section to an expansion diameter corresponding to the morphological descriptor m_{d}; and
comparing the morphological descriptor m_{d} with a nominal morphological descriptor Mₙ of the proximal section:
a point Pₐ being defined as Pₐ=P_{d};
identifying a point P_{dm} that is located an interval m_{d} from the distal point P_{d} and on a plane perpendicular to and intersecting the traced central line at the distal point P_{d},
if the morphological descriptor m_{d} is smaller than the nominal morphological descriptor Mₙ, the method further comprises:
vii. making the point Pₐ equal to a point next to Pₐ in a proximal direction along the traced central line,
viii. calculating dₐ as the distance between the point Pₐ and the point P_{dm}, and
iv. comparing the calculated distance dₐ with the distance h_{d}:
if the distance dₐ is smaller than the distance h_{d}, the method further comprises repeating steps vii. to viii.,
if the distance dₐ is greater than or equal to the distance h_{d}, Pₚ=Pₐ is defined as the proximal point that limits a portion of the central line over the traced central line that will be needed for deploying the proximal section; or
if the morphological descriptor m_{d} is greater than or equal to the nominal morphological descriptor Mₙ, the method comprises selecting the proximal point Pₚ as the point that is located a distance dₘᵢₙ from the distal point P_{d} in the proximal direction, where dₘᵢₙ is a minimum height, over the traced central line, achieved by the proximal section, corresponding to a height of the proximal section being in a configuration corresponding to the morphological descriptor m_{d}.

2. The method according to claim 1, wherein the interlaced device includes a singularity in the proximal section and a singularity in the distal section, a calculation of the proximal and distal ends of the interlaced device is performed for both the proximal section and the distal section, being the distal point P_{d} of the proximal section defined as the proximal point Pₚ of the distal section.

3. The method according to claim 2, wherein the interlaced device further comprises a central section, the traced central line of the vascular structure is divided into different segments, the method further comprising:
x. selecting a point P_{c} of the traced central line at which deployment of the interlaced device of the distal section ends, the point P_{c} is the proximal point Pₚ of the distal section;
xi. extracting from the traced central line at least one morphological descriptor m_{c} of the segment corresponding to point P_{c};
xii. calculating a height of the interlaced device for a first segment using a ratio indicating a change in height of the interlaced device according to the local morphology of the vascular structure;
xiii. subtracting the calculated height from a nominal height of the interlaced device, obtaining a new nominal height,
if the new nominal height is greater than 0, the method further comprises repeating steps xi. to xiii. for a segment contiguous to a preceding segment, moving forward in the proximal direction, and if the new nominal height is approximately 0, all lengths of each segment are added together, a result of the addition being a final height of the interlaced device after its positioning.

4. The method according to claim 1, further comprising:
attaching the interlaced device to a second interlaced device at either the proximal end or distal end thereof of the first interlaced device, the first interlaced device comprising a substantially equivalent size as the second interlaced device, and the second interlaced device comprising a singularity in at least one of a proximal section or a distal section of the second interlaced device; and
performing a calculation of the proximal end and distal ends of the second interlaced device for at least one of the proximal section or distal section of the second interlaced device.

5. The method according to claim 1, wherein the morphological descriptor m_{d} comprises: a minimum radius of the cross-section of the vascular structure perpendicular to the central line, or a maximum radius, or a radius of an equivalent circumference with a perimeter equal to the cross-section of the vascular structure perpendicular to the central line, or a radius of an equivalent circumference with an area equal to the cross-section of the vascular structure perpendicular to the central line.

6. The method according to claim 1, wherein the local morphological descriptor mₐ comprises: a minimum radius of the cross-section of the vascular structure perpendicular to the central line, or a maximum radius, or a radius of an equivalent circumference with a perimeter equal to the cross-section perpendicular to the central line, or a radius of an equivalent circumference with an area equal to the cross-section perpendicular to the central line.

7. The method according to claim 1, further comprising:
dividing a surface of the singularity into a specific number of portions, the number of portions comprising a common center at the distal point P_{d}, covering the entirety of the surface and being dependent on the number of interlaced threads constituting the interlaced device;
dividing the surface into concentric circumferences, the circumferences comprising a common center at the distal point P_{d};
dividing the surface into a plurality of cells, each cell is being obtained considering a section of one of the portions contained between two consecutive concentric circumferences;
calculating for each of the plurality of cells: a total area, an area of a thread going through the cell, and an uncovered area; and
calculating a porosity of each cell as:
a ratio between the uncovered area divided by a total area of the cell; or
a ratio between the covered area divided by a total area of the cell.

8. The method according to claim 7, wherein all of the portions have an equal size.

9. The method according to claim 7, wherein all of the portions have a different size.

10. The method according to claim 7, wherein the number of portions is equal to the number of interlaced threads.

11. A non-transitory computer program product including code instructions which, when implemented in a processor of a computing device, implement a method for calculating proximal and distal ends of an interlaced device before being positioned in a vascular structure, by:
using a computer to receive a three-dimensional image of a vascular structure in which a device formed by interlaced threads, also termed interlaced device, will be positioned, and tracing a central line of the vascular structure in the three-dimensional image defining a direction in which the interlaced device is to be deployed, the interlaced device comprising a proximal end disposed at a proximal section thereof, the proximal end comprising a singularity, the singularity comprising a coincidence of a plurality of threads, or the interlaced device comprising a distal section disposed at a distal end thereof, the distal section comprising the singularity;
using the computer to define, based on an input provided by a user, a distal point P_{d} on the traced central line and a local morphology of a vessel, the distal point P_{d} being configured to indicate a point where the distal end will start to be deployed;
using the computer to calculate a proximal point Pₚ by using the defined distal point P_{d} and the defined local morphology of the vessel, the proximal point Pₚ being configured to indicate a point that limits a portion of the central line over the traced central line that will be needed for deploying the proximal section comprising the singularity or that will be needed for deploying the distal section comprising the singularity:
if the distal section comprises the singularity, the method further comprises extracting a morphological descriptor m_{d} of the vascular structure at the distal point P_{d} and comparing the morphological descriptor m_{d} with a nominal morphological descriptor Mₐ of the distal section:
a point Pₐ is defined as Pₐ=P_{d};
if the morphological descriptor m_{d} is smaller than the nominal morphological descriptor Mₙ, the method further comprises:
i. making the point Pₐ equal to a point next to Pₐ in a proximal direction along the traced central line,
ii. calculating a local morphological descriptor mₐ of a cross-section of the vascular structure at the point Pₐ,
iii. calculating a distance hₐ as hₐ=Mₙ·dumping (mₐ),
where dumping(m) is a mathematical function in an interval [0,1] which considers a variation of hₐ according to an expansion of the distal section to an expansion diameter corresponding to the local morphological descriptor mₐ,
iv. identifying a point Pₐₘ that is located an interval hₐ from the distal point P_{d} and on a plane perpendicular to and intersecting the traced central line at point Pₐ,
v. calculating d_{d} as the distance between the point Pₐ and the point Pₐₘ, and
vi. comparing the calculated distance d_{d} with the local morphological descriptor mₐ:
if the distance d_{d} is smaller than the local morphological descriptor mₐ, steps i. to v. are repeated,
if the distance d_{d} is greater than or equal to the local morphological descriptor mₐ, Pₚ=Pₐ is defined as the proximal point that limits a portion of the central line over the traced central line that will be needed for deploying the distal section; or
if the morphological descriptor m_{d} is greater than or equal to the nominal morphological descriptor Mₙ, the method comprises selecting the proximal point Pₚ as the point that is located a distance dₘᵢₙ from the distal point P_{d} in the proximal direction, where dₘᵢₙ is a minimum height, over the traced central line, defined by the distal section, and corresponding to a height of the distal section being in a configuration corresponding to the nominal morphological descriptor Mₙ; or
if the singularity is at the proximal end, the method further comprises:
extracting a morphological descriptor m_{d} of the vascular structure at the distal point P_{d};
calculating a distance h_{d} as h_{d}=Mₙ≯dumping (m_{d}),
where dumping(m) is a mathematical function an interval [0,1] which considers a variation of h_{d} according to an expansion of the proximal section to an expansion diameter corresponding to the morphological descriptor m_{d}; and
comparing the morphological descriptor m_{d} with a nominal morphological descriptor Mₙ of the proximal section:
a point Pₐ is defined as Pₐ=P_{d};
identifying a point P_{dm} that is located an interval m_{d} from the distal point P_{d} and on a plane perpendicular to and intersecting the traced central line at the distal point P_{d},
if the morphological descriptor m_{d} is smaller than the nominal morphological descriptor Mₙ, the method further comprises:
vii. making the point Pₐ equal to a point next to Pₐ in a proximal direction along the traced central line,
viii. calculating dₐ as the distance between the point Pₐ and the point P_{dm}, and
iv. comparing the calculated distance dₐ with the distance h_{d}:
if the distance dₐ is smaller than the distance h_{d}, the method further comprises repeating steps vii. to viii.,
if the distance dₐ is greater than or equal to the distance h_{d}, Pₚ=Pₐ is defined as the proximal point that limits a portion of the central line over the traced central line that will be needed for deploying the proximal section; or
if the morphological descriptor m_{d} is greater than or equal to the nominal morphological descriptor Mₙ, the method comprises selecting the proximal point Pₚ as the point that is located a distance dₘᵢₙ from the distal point P_{d} in the proximal direction, where dₘᵢₙ is a minimum height, over the traced central line, achieved by the proximal section, corresponding to a height of the proximal section being in a configuration corresponding to the morphological descriptor m_{d}.

12. The method according to claim 3, further comprising:
attaching the interlaced device to a second interlaced device at either the proximal end or distal end of the first interlaced device, the first interlaced device comprising a substantially equivalent size as the second interlaced device, and the second interlaced device comprising a singularity in at least one of a proximal section or a distal section of the second interlaced device; and
performing a calculation of the proximal end and distal end of the second interlaced device for at least one of the proximal section or distal section of the second interlaced device.
